(19) United States
(12) Patent Application Publication
(10) Pub. No.: US 2025/0327029 A1
(43) Pub. Date: Oct. 23, 2025

(54) PROCESS FOR THE OBTENTION OF INVARIANT NATURAL KILLER T CELLS

(71) Applicants: Université de Lorraine, Nancy (FR); Centre National de la Recherche Scientifique CNRS, Paris (FR); Centre Hospitalier Régional Universitaire de Nancy, Nancy (FR)

(72) Inventors: Marie Thérèse Rubio, Nancy (FR); Jordan Brouard, Vandoeuvre les Nancy (FR)

(73) Assignees: Université de Lorraine, Nancy (FR); Centre National de la Recherche Scientifique CNRS, Paris (FR); Centre Hospitalier Régional Universitaire de Nancy, Nancy (FR)

(21) Appl. No.: 18/870,062

(22) PCT Filed: May 29, 2023
(86) PCT No.: PCT/EP2023/064344

§ 371 Date: Nov. 27, 2024

(30) Foreign Application Priority Data
May 30, 2022 (EP) 22305788.6

(51) Int. Cl. C12N 5/0783

(52) U.S. Cl. CPC C12N 5/0646; C12N 2500/32; C12N 2500/36; C12N 2500/84; C12N 2501/2315; C12N 2506/11

(57) ABSTRACT

The present invention pertains to cell culture protocols and particularly provides a method for obtaining Invariant Natural Killer T (iNKT) cells. The method according to the present invention particularly comprises culturing peripheral blood mononuclear cells (PBMC) in a culture medium comprising alpha-Galactosylceramide (alpha-GalCer) and Interleukin 15 (IL-15). The present invention allows for the specific expansion of CD4V iNKT which have a particular interest in the prevention of graft versus host disease in the context of hematopoietic stem cell transplantation.

### TECHNICAL FIELD

The present invention pertains to the field of cell culture. The present invention particularly relates to the culture and expansion of human invariant natural killer (iNKT) cells.

### BACKGROUND ART

Hematopoietic stem cell (HSC) allograft remains the only potentially curative treatment for certain hematologic malignancies, particularly acute leukemias. Allogeneic transplantation is an immunotherapy that aims at eliminating residual tumor cells in a patient by using the donor's immune system (GVL effect for Graft versus Leukemia). Unfortunately, this GVL effect is often accompanied by graft versus host disease (GVHD). GVHD occurs when the donor's T cells (from the graft) view the patient's healthy cells (the host) as foreign, attack and damage them. GVHD can be fatal and is the first complication of HSC allograft transplantation (occurs in 30 to 50% of cases). The first-line treatment for GVHD is corticosteroid therapy. However, this therapy is accompanied by numerous deleterious side effects such as diabetes, osteoporosis, hypertension, severe infections and risk of leukemia relapse. Furthermore, 50% of patients have a cortico-refractory GVHD and these patients have a very compromised life expectancy (<50% survival at one year).

The present inventors have observed that patients transplanted with an allogeneic HSC graft have a highly variable post-transplant reconstitution of invariant NKT (iNKT) lymphocytes, and that patients with a good reconstitution (i.e. superior to the median iNKT level in healthy subjects) in the first 3 months after transplantation have a better overall survival after transplantation due to a reduced incidence of acute GVHD with no increased risk of relapse of the underlying malignant disease (see Rubio et al., Blood, The Journal of the American Society of Hematology 120.10 (2012): 2144-2154).

Furthermore, the present inventors have also shown that patients receiving HSC allografts with high iNKT levels and good ex vivo expansion of CD4⁻ iNKT are protected from the risk of acute GVHD and have a better post-transplant reconstitution of iNKT cells (Rubio et al., Leukemia 31.4 (2017): 903-912). These results confirmed the observations made by Chaidos et al (Blood, The Journal of the American Society of Hematology 119.21 (2012): 5030-5036), showing a correlation between the level of iNKT, in particular CD4⁻ iNKT, in peripheral blood stem cell grafts and the occurrence of acute GVHD.

Finally, the present inventors have also shown that in a pre-clinical xeno-GVHD mouse model (human peripheral blood mononuclear cells [PBMC]transplanted into NSG immunodeficient mice), the CD4⁻ subtype of human iNKT—unlike the CD4⁺ subtype—is able to regulate GVHD by destroying dendritic cells (mediators of GVH) via direct cytotoxicity and inhibiting the maturation of these antigen-presenting cells (APC) and consequently inhibiting the activation of human conventional T cells responsible for GVHD (Coman et al., Oncoimmunology 7.11 (2018): e1470735).

Other teams have reported reduced risk of relapse in patients presenting better iNKT reconstitution after allograft (De Lalla et al., The Journal of Immunology 186.7 (2011): 4490-4499), or improved relapse-free survival with no severe GVHD in patients who received iNKT-rich grafts (Malard et al., Blood, The Journal of the American Society of Hematology 127.14 (2016): 1828-1835).

All these results suggest that iNKT lymphocyte enrichment of HSC grafts could control GVHD without altering the GVL effect after transplantation.

However, the level of iNKT lymphocytes is very variable from one subject to another (ranging from 0.001% to 1% of T lymphocytes) and their use in cellular immunotherapy requires the development of a clinical-grade protocol for the expansion of iNKT cells, and particularly of the CD4⁻ subtype.

iNKT lymphocytes can be expanded in vitro by culturing PBMC in RPMI medium, 10% SVF, in the presence of alpha-GalCer (a synthetic glycolipid which induces the activation of iNKT cells via their invariant T receptor that recognizes glycolipids presented by the CD1d molecule expressed on the surface of antigen-presenting cells such as dendritic cells and monocytes) and interleukin 2 (IL-2). Although this classical expansion protocol allows for the expansion of CD4⁺ iNKT cells, it does not allow obtaining sufficient quantities of CD4⁻ iNKT cells. Such a protocol is useful for experimentally studying the functionality of iNKTs, but not for obtaining CD4⁻ cells that can be used in the context of HSC transplantation.

Alternatively, it is possible to expand iNKTs by first sorting iNKTs from PBMCs and then culturing them with APCs (dendritic cells or PBMCs) in the presence of IL-2 and alpha-GalCer. This technique does not allow promoting CD4⁻ iNKT expansion either, and is not compatible with clinical applications.

One biotech company (MiNK Therapeutics) has developed iNKT expansion projects in the context of GVHD (and anti-cancer immunotherapy). Their protocol comprises sorting iNKT cells before expansion using either an irradiated cell line or irradiated PBMCs as alpha-Gal-Cer-presenting cells to expand iNKTs in the presence of alpha-Gal-Cer and IL-2 (Exley et al., Clinical Cancer Research 23.14 (2017): 3510-3519). The iNKT cells thereby obtained are mostly CD4+ iNKT cells.

The generation of CAR-iNKT cells (human iNKT transduced with a chimeric antigen receptor) has also been reported (international patent application published under reference WO2019/166817). iNKTs are first sorted, expanded in the presence of irradiated PBMCs during the transfection phase, and then the transduced cells are expanded in the presence of an irradiated transduced CD1d line. This technique is cumbersome and does not allow for the efficient promotion of CD4⁻ iNKT cells.

There is thus an urgent need for cell culturing protocols that allow for the obtention of iNKT cells, in particular CD4⁻ iNKT cells, in conditions that are compatible with clinical applications. The provision of such protocols is essential to obtain high quality HSC grafts that will allow for an efficient treatment of hematological malignancies with limited risks of GVHD.

### SUMMARY

The invention is defined by the claims.

The present inventors have developed an expansion technique that meets the required quality standards (GMP) for clinical-grade culture conditions and allows for the preferential expansion of human CD4⁻ iNKTs which can advantageously be used in the prevention of acute GVHD after HSC allograft. This technique is based on culturing PBMCs in presence of Galactosylceramide (alpha-GalCer) and human Interleukin 15 (IL-15).

Thus, the present invention pertains to a method for obtaining iNKT cells, said method comprising a step of culturing peripheral blood mononuclear cells in a culture medium comprising alpha-GalCer) and Interleukin 15 (IL-15).

This method is the only approach that allows for the expansion of human CD4⁻ iNKT at high rates, with an expansion factor between 10 and 10 000 within 14 days of culture. The method according to the present invention particularly allows for the obtention of more than 100×10⁶ iNKT CD4⁻ cells from a lymphapheresis containing around 109 PBMCs. These extremely promising results are compatible with high-grade clinical application and represent an extremely promising therapeutic tool in the context of HSC transplantation.

The method according to the present invention can particularly be performed under GMP culture conditions and requires very few manipulations during the culturing process. It indeed requires at most the provision of total PBMC, addition of cytokines, optionally partially replacing the culture medium once or twice and finally sorting/isolating iNKT cells at the end of the culture. The present method is thus simple, easily reproducible, and extremely efficient for obtaining a high number of CD4⁻ iNKT cells within a short period of time.

According to a preferred embodiment, the method according to the present invention advantageously comprises a step of screening and isolating CD4⁻ iNKT cells.

IL-15 is typically introduced at a concentration of around 10 ng/mL in the culture medium, and alpha-GalCer at a concentration of around 100 ng/mL.

A further aspect of the invention pertains to the use of a combination of alpha-GalCer and IL-15 for obtaining iNKT cells, preferably CD4⁻ iNKT cells, from PBMC.

### BRIEF DESCRIPTION OF DRAWINGS

FIG. 1: Expansion of iNKT cells from healthy donor PBMC in presence of IL-15. A. Dot plots obtained by flow cytometry of total iNKT cells: cells co-expressing CD3 and iNKT invariant T-cell receptor [TCR](% among total T lymphocytes). B. Histograms showing the distribution of CD4⁻ and CD4⁺ subtypes among the total iNKT population obtained. At DO, PBMCs contained 0.82% iNKT cells of which 61% were CD4⁻ and 38% CD4⁺. The percentage of iNKT cells at the end of the 14-day culture increased to 81.5% with a predominance of CD4⁻ iNKT cells (61% among the expanded iNKT cells).

FIG. 2: CD4⁻ iNKT expansion factor according to the cytokine used. A. Comparison of CD4⁻ iNKT expansion factor in the presence of IL-2 or IL-15 during the expansion phase. The expansion factor was significantly increased in the presence of IL-15 in comparison to IL-2 (mean expansion factor of CD4⁻ iNKT cells±SEM: 652±128 with IL-15 vs 158±38 with IL-2, p=<0,0001)(n=17). B. The proportion of CD4⁻ iNKT cells is increased at D14 of culture in the presence of IL-15 in comparison to IL-2 (mean % CD4⁻ iNKT cells±SEM: 60.6±5.23 with IL-15 vs 47.7±7.35 with IL-2, p=0.0010), (n=14).

FIG. 3: CD4⁻ iNKT cell expansion factor and number of CD4⁻ iNKT cells obtained according to the method of the present invention using medium and large volumes of culture. PBMCs are cultured at a concentration of 1×10⁵/mL to 10⁶/mL in the presence of alphaGalCer and IL-15 for 14 days. PBMCs are concentrated at 10⁶/mL in culture bags (corresponding to medium volumes of culture) and 1 (D1) to 2 (D2)×10⁵/mL in bioreactor (corresponding to large volumes of culture). A. Expansion factor of CD4⁻ iNKT cells in different culture conditions (all according to the invention). The expansion factor varies from 10 to 1000 when cultures are performed in bags and between 100 and 10 000 when cultures are performed in bioreactors. A 10-fold increase expansion factor has been observed between bag and bioreactor from PBMC from the same donor in 5 separate experiments (comparison between 100 mL bag and D2 bioreactor by Kruskal Wallis test: p-value=0.029). B. Number of CD4⁻ iNKT cells at day 0 (DO) and day 14 (D14) of culture in different conditions. The mean number of CD4⁻ iNKT cells obtained at the end of the culture was 4.67×10⁶ (+/−SEM 3.5×10⁶) in a 100 mL bag from 100×10⁶ PBMCs in comparison to 14.85×10⁶ (+/−SEM 7.5×10⁶) in a 100 ml bioreactor from 10×10⁶ PBMCs (see FIG. 3B) and to 27.5×10⁶ (+/−SEM 21.3×10⁶) in a 100 ml bioreactor from 20×10⁶ PBMCs (see FIG. 3B). The method according to the present invention therefore allows for an extremely efficient CD4⁻ iNKT cell expansion regardless the volumes of culture used.

FIG. 4: iNKT expansion factors and numbers of CD4⁻ iNKT obtained according to the method by varying the number of iNKT cell concentration at day 0 or by prolongating the expansion up to 21 days. A. PBMCs are cultured at a concentration of 2×10⁵/mL (2M/cm2) or at a concentration of 10 000 iNKT/cm2 or 1000/mL) in bioreactors in the presence of alphaGalCer and IL-15 for 14 days. Expansion factor of total, CD4⁻ and CD4+ iNKT cells were not improved in 3 cultures by concentrating iNKT cells at day at 10000/cm2 in comparison to 2M PBMCs/cm2. B. PBMCs are cultured at a concentration of 2×10⁵/mL in bioreactors in the presence of alphaGalCer and IL-15 for 21 days with (BC45 and 46) or without (BC23 and 42) restimulation with PBMCs in the presence of alphaGalCer and IL-15 at day 14. The numbers of CD4⁻ iNKT cells obtained at 21 were not increased by prolongating the expansion duration.

FIG. 5: Modulation of GVHD by human expanded iNKT cells in a humanized xeno-GVHD model. NSG™ mice were irradiated (2 Gy total body irradiation) on day −1 and transplanted with 5×10⁶ human PBMC on day 0, without (PBMC alone group, n=4) or with 10⁶ human iNKT cells expanded as defined in the present invention (PBMC+iNKT group, n=9). At day 60, all mice in the PBMC group have died of GVHD, while 5/9 mice in the PBMC+iNKT group were alive without GVHD.

### DESCRIPTION OF EMBODIMENTS

The present invention thus pertains to a method for obtaining human Invariant Natural Killer T (iNKT) cells, said method comprising a step of culturing peripheral blood mononuclear cells (PBMC) in a culture medium comprising alpha-Galactosylceramide (alpha-GalCer) and Interleukin 15 (IL-15), wherein the expansion factor of iNKT cells is equal to or higher than 10, preferably 100, more preferably 500. The expansion factor of iNKT cells is more preferably comprised between 100 and 10 000.

Invariant Natural Killer T (iNKT) cells are a T cells subtype characterized by the expression of a semi-invariant TCR (T cell receptor) which, contrary to the TCR expressed by other T cells subtypes which mainly interacts with peptides presented by MHC molecules, recognizes glycolipids presented by CD1d, a non-polymorphic MHC-1-like molecule. iNKT cells contribute to anti-infective responses but also exhibit immunosuppressive functions. They can produce several cytokines upon stimulation, such as IL-4 and IFN-γ, and express the natural killer (NK) cell marker NK1.1 in mice and are mainly defined as being CD3′ and invariant TCR⁺ in humans. iNKT cells require CD1d expression for their development and bounding of CD1d to the glycolipid Alpha-GalCer, strongly stimulates iNKT expansion. iNKT cells in humans can be broadly categorized as either CD4⁻/CD8⁻ cells (double negative), CD4⁺, and a small percentage of CD8+ cells (see for review Krovi, S. Harsha, and Laurent Gapin, Frontiers in immunology 9 (2018): 1393). As mentioned above, the specific subpopulation of CD4⁻ iNKT cells can regulate GVHD by destroying dendritic cells which are known to mediate GVHD. The specific CD4⁻ iNKT subtype is thus an extremely promising tool for the prevention of GVHD in the context of HSC transplantation. The method according to the present invention, by specifically using IL-15 in the culture medium, allows for the advantageous expansion, in other words multiplication and maintenance, of this specific subtype in the culture medium.

Advantageously, the method according to the present invention can comprise a step of screening and isolating CD4⁻ iNKT cells at the end of the culturing step. Such a screening step advantageously allows for the specific obtention of CD4⁻ iNKT cells. Nevertheless, given the fact that the method according to the present invention allows for the efficient multiplication and maintenance of CD4⁻ iNKT cells, such a screening step does not necessarily have to be performed: the total population of iNKT cells obtained at the end of the present method can directly be used in therapeutic applications.

iNKT cells can be isolated from other cell populations via several techniques known by the skilled person (see e.g. Exley et al., Current protocols in immunology 90.1 (2010): 14-11; or Watarai, Hiroshi et al. Nature protocols 3.1 (2008): 70-78). These techniques include e.g. using immunoaffinity columns (including fluorescence-activated cell sorting/FACS), flow cytometry, immunomagnetic techniques and magnetic beads. According to a preferred embodiment, CD4⁻ iNKT cells can be isolated by using magnetic cell isolation techniques. The skilled person is completely familiar with all these techniques and several kits and apparatus are currently available for performing them. One preferred technique consists in performing a magnetic CD4⁺ cell isolation (e.g by using the CD4 MicroBeads kit commercialized by Miltenyi Biotec Inc.), retrieve the negative fraction, performing a magnetic iNKT⁺ isolation on said fraction (e.g. by using the Anti-iNKT MicroBeads kit commercialized by Miltenyi Biotec Inc.) and retrieve the positive fraction corresponding to the isolated CD4⁻ iNKT cell population.

According to the present invention, iNKT cells are obtained from an inoculum of peripheral blood mononuclear cells (PBMC) into the culture medium. PBMC include lymphocytes (T cells, B cells, NK cells), monocytes and a minority of dendritic cells. The skilled person is completely familiar with techniques allowing for the obtention of PBMCs from a patient. These cells can e.g. be extracted from whole blood samples using a hydrophilic polysaccharide such as ficoll that separates layers of blood, and gradient centrifugation. PBMCs are generally collected by continuous-flow apheresis or lymphapheresis from a donor. In the context of the present invention, PBMCs are preferably obtained from healthy patient, i.e. a healthy donor. The donor does thus not suffer from any illness. According to this embodiment, the iNKT cells obtained according to the method of the present invention are used for allogenic transplantation.

PBMC are preferably inoculated/introduced in the culture medium at a concentration ranging from 1×10⁴ to 1×10⁷ cells/mL, preferably from 1×10⁵ to 2×10⁶ cells/mL.

The “culture medium” used in the context of the present invention is any physiologically acceptable medium that allows for the growth of iNKT cells. Physiologically acceptable media are aqueous media that comprise electrolytes such as sodium potassium, magnesium and/or calcium salts, including anions such as chloride, carbonate, hydroxide or caprylate. Suitable culture medium according to the present invention typically comprise D-Glucose, non-essential amino-acids, sodium pyruvate, ethanolamine, glutathione, ascorbic acid, insulin, transferrin, albumin, sodium bicarbonate, sodium chloride, sodium phosphate dibasic, magnesium sulfate, potassium chloride, calcium nitrate and/or L-Arginine. A suitable culture medium that can be used for performing the method according to the present invention is e.g. the RPMI (Roswell Park Memorial Institute) 1640 advanced medium commercialized under reference Gibco® by Thermo Fisher Scientific. Such a culture medium typically comprises glucose, non-essential amino-acids, sodium pyruvate, phenol red, ethanolamine, glutathione, ascorbic acid, insulin, transferrin and lipid-rich bovine serum albumin as well as the following trace elements: sodium selenite, ammonium metavanadate, cupric sulfate and manganese chloride. According to a specific embodiment, the culture medium according to the present invention can also comprise fetal bovine serum (FBS) and/or human AB serum and/or glutamine. FBS, human AB serum and glutamine are conventionally used for in vitro cell culture of eukaryotic cells and can be easily obtained from specialized manufacturers. According to this embodiment, the concentration of FBS or human AB serum in the culture medium is typically comprised between 1 and 8% v/v, for example between 1 and 5% v/v, preferably around 5% v/v (i.e. between 4.8 and 5.2%), more preferably of 5% v/v, and the concentration of glutamine is comprised between 1 and 10 mM, preferably around 4 mM (i.e. between 3.8 and 4.2 mM), more preferably of 4 mM.

The method according to the present invention comprises culturing PBMCs with human IL-15. The present inventors have indeed demonstrated that the use of this specific cytokine strongly promotes the expansion, i.e. multiplication, of the specific CD4⁻ subtype of iNKT cells in the culture medium. The present inventors have shown that no cytokine other than IL-15 is necessary for the expansion of iNKT cells in the context of the present invention. Therefore, according to a preferred embodiment, the culture medium used in the context of the present invention does not comprise any other cytokine than IL-15. In other words, IL-15 is the only cytokine used for obtaining iNKT cells from PBMCs in the context of the present invention.

Interleukin 15 (IL-15 or IL15) is a cytokine that stimulates the proliferation of T-lymphocytes. Stimulation of T cells by IL15 generally results from the interaction of IL15 with components of the IL2 receptor, including IL2RB. IL-15 signaling in neutrophils stimulates phagocytosis. It can be easily obtained from specialized manufacturers and has an amino acid sequence accessible under reference P40933 in the Uniprot database.

The concentration of IL-15 in the culture medium is typically comprised between 5 and 15 ng/mL, preferably between 8 and 12 ng/mL, more preferably around 10 ng/mL (i.e. comprised between 9.5 and 11.5 ng/mL). According to a specific embodiment, the concentration of IL-15 in the culture medium is 10 ng/mL.

The method according to the present invention according to the present invention also comprises culturing the PBMCs in the presence of alpha-GalCer.

Alpha-Galactosylceramide (alpha-GalCer or α-GalCer) is a synthetic glycolipid that has the ability to activate iNKT cells. Alpha-GalCer interacts with cD1d and the invariant TCR of iNKT cells is able to bind to the CD1d/alpha-GalCer complex thereby leading to iNKT cell activation in both mice and humans. Alpha-GalCer has the chemical formula C₅₀H₉₉NO₉ and is accessible under reference 2826713 in the PubChem library. It can be easily obtained from specialized manufacturers.

The concentration of alpha-GalCer in the culture medium is typically comprised between 50 and 150 ng/mL, preferably between 80 and 120 ng/mL, more preferably around 100 ng/mL (i.e. comprised between 95 and 105 ng/mL). According to a specific embodiment, the concentration of alpha-GalCer in the culture medium is 100 ng/mL.

The method according to the present invention is characterized by the fact that the expansion factor of iNKT cells is equal to or higher than 10, preferably 100, more preferably 500. The expansion factor of iNKT cells is more preferably comprised between 100 and 10 000. The “expansion factor” corresponds to the number of iNKT cells obtained at day 14 of expansion divided by the number of iNKT cells put in culture at day 0. The number of cells is determined by routinely used techniques by the skilled person (cell couting and flow cytometry staining to determine the percentage of iNKT cells among total PBMCs).

The present invention thus also pertains to the use of a combination of alpha-GalCer and IL-15 for obtaining iNKT cells from PBMC, preferably CD4⁻ iNKT cells.

In the context of the present invention, the culture of the PBMCs is typically performed under normoxic conditions i.e. in physiological oxygen concentrations. The cells are thus cultured in the presence of 5% of carbon dioxide (CO₂).

The culture of the PBMCs is typically performed at physiological temperature, i.e at a temperature comprised between 36 and 38° C., preferably 37° C.

The method according to the present invention allows obtaining iNKT cells, in particular CD4-iNKT cells within 14 days. Thus, according to a specific embodiment, the culturing step is performed over 7 to 21 days, preferably over 12 to 16 days, more preferably over 14 days.

The culturing step can be carried out by placing the culture medium in a cell culture bag. Cell culture bags are routinely used by the skilled person who can easily obtain them from various manufacturers. Alternatively, the culturing step can also be carried out by placing the culture medium in a bioreactor. The use of a bioreactor allows culturing larger volumes of culture medium and further allows significantly increasing the cell expansion factor. The bioreactor can be any bioreactor allowing for the growth and expansion of cells, in particular of PBMCs and iNKT cells. The bioreactor is typically a continuous reactor. Bioreactors useful in the context of the present invention are plentiful and can be easily obtained. One can e.g. cite the G-Rex® produced and commercialized by Wilson Wolf Manufacturing, and which is a cell culture flask with a gas-permeable membrane at the base that supports large media volumes without compromising gas exchange.

As disclosed above, alpha-GalCer is used for promoting the activation of iNKT cells among the PBMC and IL-15 allows promoting the expansion (multiplication and maintenance) of the specific CD4⁻ iNKT subtype among the total iNKT cell population obtained after activation by alpha-GalCer.

In the context of the present invention, PBMCs can therefore be directly cultured in presence of both alpha-GalCer and IL-15. According to such an embodiment, the culture medium already comprises both alpha-GalCer and IL-15 when PBMCs are added, i.e. at day 0 of culture. According to this embodiment, the method for obtaining iNKTs can be divided into two main phases: 1/activation and expansion phase of the iNKTs and 2/final sorting phase wherein CD4⁻ iNKT cells are isolated from the culture medium.

Alternatively, PBMCs can also be cultured in presence of alpha-GalCer before adding IL-15 into the culture medium. IL-15 can e.g. be introduced into the culture medium after 1 to 5 days of culture of the PBMCs in presence of alpha-GalCer, preferably after 1 day of culture. According to this specific embodiment, the method for obtaining iNKT cells can be divided into three main phases: 1/An activation phase of the iNKTs, 2/An expansion phase and 3/a final sorting phase wherein CD4-iNKT cells are isolated from the culture medium.

The method according to the invention can be easily put into practice. In particular, the method according to the invention can advantageously be simply carried out by culturing PBMCS on a culture medium comprising alpha-GalCer and IL-15 over 7 to 21 days, preferably over 14 days.

According to such an embodiment, the method according to the invention can comprise the following steps: (i) PBMC are cultured on the culture medium (such as e.g. the RPMI Advanced culture medium+2 or 5% FBS), preferably at a concentration of 2×10⁵ cells/mL with IL-15, alpha-GalCer and optionally FBS, human AB serum and/or glutamine; (ii) on day 14 of the culture, iNKT cells are sorted.

Alternatively, the culture medium can be replaced with a new culture medium after a few days of culture. The culture medium used as a replacement (also referred to as “new” culture medium) is as defined above and is typically the same as that used in the preceding steps of the method, i.e. as the culture medium that is being replaced. Said replacement can affect 100% of the culture medium, i.e. the totality of the culture medium used in the preceding steps is replaced with the new culture medium, or, alternatively, the culture medium is only partially replaced, and, in that case, 20 to 80%, preferably 40 to 60%, more preferably 50% of the culture medium can be replaced with the new culture medium. Such a replacement is generally performed once or several times (such as 2, 3, 4 or 5 times) during the expansion phase, i.e. the phase during which the population of iNKT cells increases or is at least maintained. According to this embodiment, said step of replacing the culture medium can be performed after 6 to 8 days, preferably after 7 days of culture and/or after 9 to 11 days, preferably after 10 days of culture.

According to this alternative embodiment, IL-15 can advantageously be added in said new culture medium. In such a case, the final concentration of IL-15 in the culture medium is as defined above.

According to such a specific embodiment the method according to the present invention can comprise the following steps:
a. PBMC are cultured on the culture medium (such as e.g. the RPMI 1640 advanced medium), preferably at a concentration of 5×10⁵ cells/mL, with alpha-GalCer and optionally FBS, human AB serum and/or glutamine;
b. on day 1 of culture, IL-15 is added to the culture medium;
c. on day 7 of culture, 50% of the medium is replaced and IL-15 is added to the culture medium;
d. on day 10 of culture, 50% of the medium is replaced and IL-15 is added to the culture medium;
e. on day 14 of the culture, iNKT cells are sorted.

According to this specific embodiment, step a) corresponds to the activation phase, step b), c) and d) correspond to the expansion phase, and step e) corresponds to the sorting phase.

As discussed above, CD4⁻ iNKT cells are able to regulate GVHD by destroying dendritic cells (mediators of GVH) via direct cytotoxicity, thereby inhibiting the maturation of these antigen-presenting cells (APC) and consequently inhibiting the activation of human conventional T cells responsible for GVHD. The iNKT cells obtained according to the method according to the present invention therefore represent an extremely powerful tool for the treatment of diseases conditions involving deleterious T-cell responses such as graft versus host disease or graft rejection, as well as autoimmune and inflammatory diseases.

“Graft versus host disease” or “GVHD” is a complication following an allogenic transplant wherein the grafted tissue cells attack the host cells. There are two forms of GVHD: acute graft versus host disease and chronic graft versus host disease (see for review Blazar al., Nature reviews immunology 12.6 (2012): 443-458).

The “patient” according to the present invention is a mammal, preferably a human.

In the context of the present invention, the iNKT cells obtained by the method disclosed above are advantageously used for preparing, in particular for enriching, a hematopoietic stem cell (HSC) graft for administration to a patient in need thereof. The administration of such an iNKT-enriched graft allows for the treatment of certain hematologic malignancies, particularly acute leukemias, while preventing/limiting the risk of GVHD.

The present invention will now be illustrated in detail in the Examples shown below. These examples are provided for illustrative purposes only. The present invention is not limited to the specific data provided in these examples.

### Examples

1. Process for the Expansion of iNKT Cells Using IL-15

### A. Culture Using Medium Culture Volumes

PBMC obtained by centrifugation of fresh whole blood on density gradient were culture at the concentration of 5.10⁵ cells/mL in RPMI 1640 advanced medium (ref: 11530446, Gibco®), with 5% SVF v/v, 4 mM glutamine, 100 ng/mL alpha-galactoside-ceramide (KRN-7000, Funakoshi). Cultures were performed in a cell culture bag in a normoxia incubator at 37° C. and 5% CO₂.

On day 1 of culture, 10 ng/mL of IL-15 were added.

On day 7 of culture, 50% of the medium was changed and 10 ng/mL of IL-15 were introduced.

On day 10 of the culture, 50% of the medium was changed and 10 ng/mL of IL-15 were introduced.

On day 14 of the culture, the obtained iNKT cells were sorted by performing a magnetic CD4⁺ cell isolation (CD4 MicroBeads kit commercialized by Miltenyi Biotec Inc.), retrieving the negative fraction, performing a magnetic iNKT⁺ isolation on said fraction (Anti-iNKT MicroBeads kit commercialized by Miltenyi Biotec Inc.) and retrieving the positive fraction corresponding to the isolated CD4⁻ iNKT cell population.

### B. Culture Using Large Culture Volumes

PBMCs were cultured at a concentration of 1 (D1) to 2 (D2)×10⁵/mL in bioreactor (G-rex®-Wilson Wolf Manufacturing) over 14 days in RPMI 1640 advanced medium (ref: 11530446, Gibco®), with 5% SVF v/v, 4 mM glutamine, 100 ng/mL alpha-galactoside-ceramide (KRN-7000, Funakoshi) and 10 ng/mL of IL-15, in normoxic conditions at 37° C. and 5% CO₂.

### 2. Results

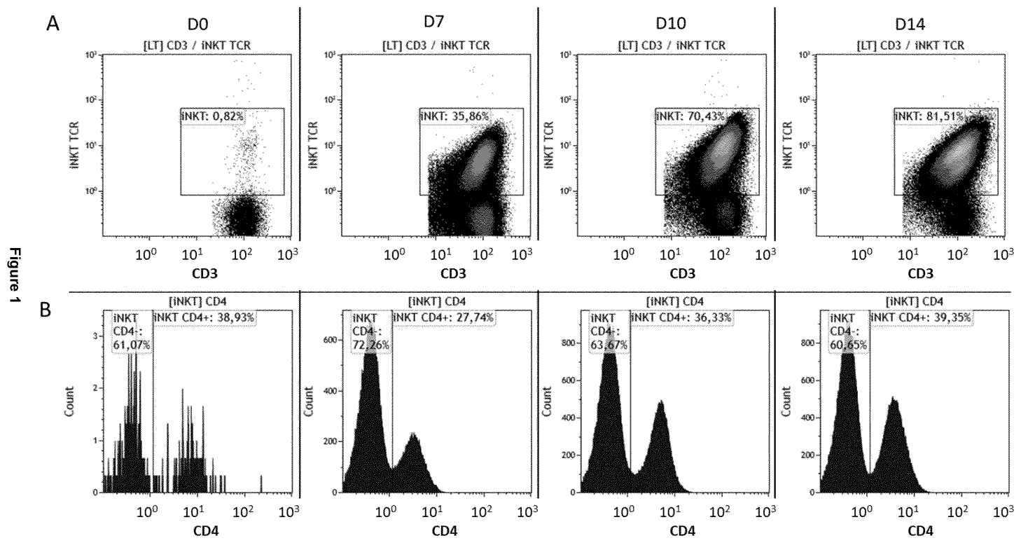

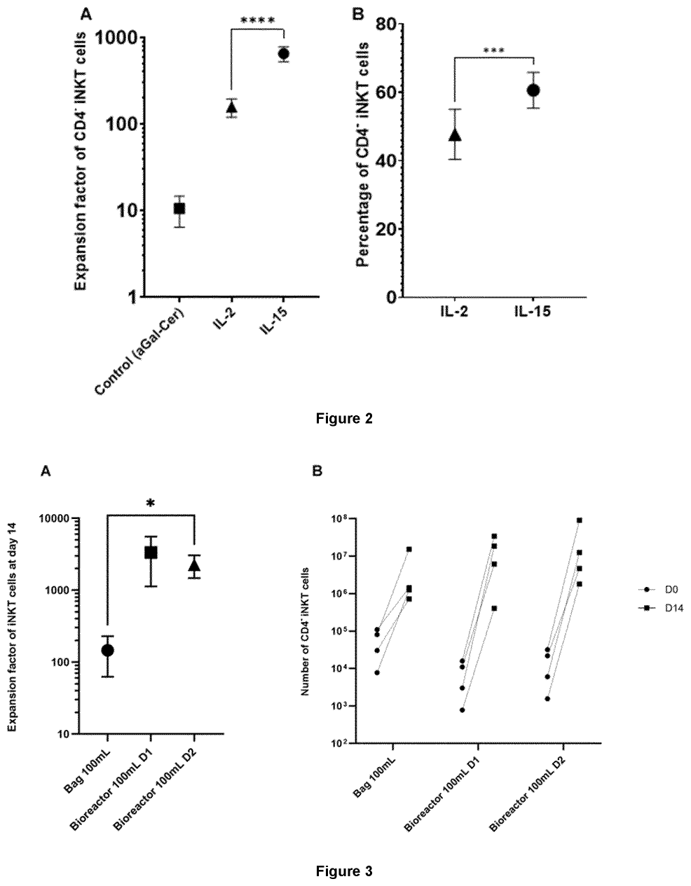

The cell culture protocol according to the invention promotes the expansion of CD4⁻ iNKTs (see FIG. 1). The results show that CD4⁻ iNKT cell expansion factor was increased in the presence of IL-15 as compared to IL-2 and IL-15, unlike IL-2, promoted CD4⁻ iNKT cell expansion (see FIG. 2).

### 3. Validation

The protocol, which had initially been optimized on small culture volumes (2 mL/well) was further validated in 100 mL culture either in bags or in a bioreactor. The expansion factor varies from 10 to 1000 when cultures are performed in bags and between 100 and 10 000 when cultures are performed in bioreactors. A 10-fold increase expansion factor has been observed between bag and bioreactor from PBMC from the same donor in 5 separate experiments (FIG. 3A). The mean number of CD4⁻ iNKT cells obtained at the end of the culture was 4.67×10⁶ (+/−SEM 3.5×10⁶) in a 100 mL bag from 100×10⁶ PBMCs in comparison to 14.85×10⁶ (+/−SEM 7.5×10⁶) in a 100 ml bioreactor from 10×10⁶ PBMCs (see FIG. 3B) and to 27.5×10⁶ (+/−SEM 21.3×10⁶) in a 100 ml bioreactor from 20×10⁶ PBMCs (see FIG. 3B). The protocol according to the present invention therefore allows for an efficient expansion of CD4⁻ iNKT cells regardless the culture volumes used.

The use of a bioreactor allows culturing large volumes of cell culture and strongly increases the expansion factor and the number of CD4⁻ iNKT cells.

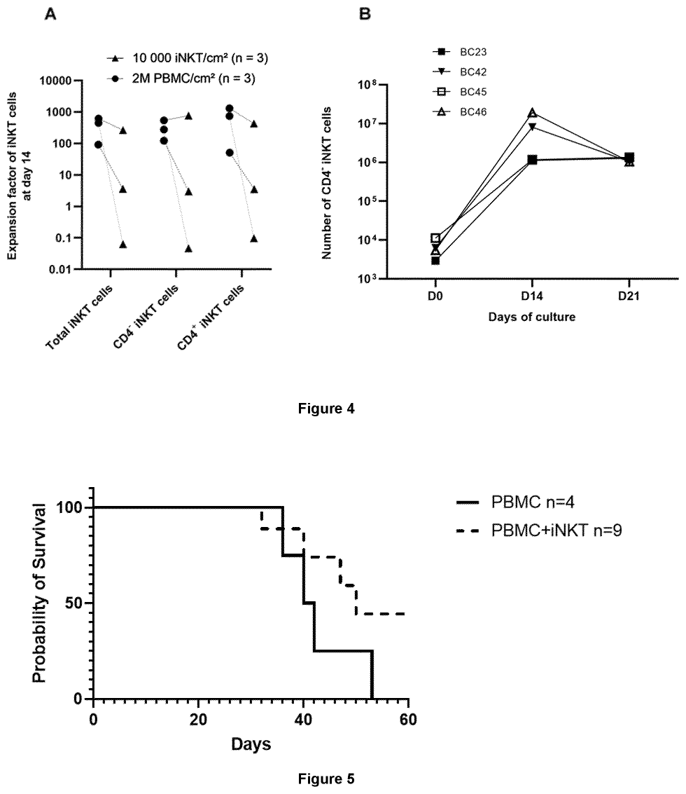

The expansion protocol was not improved by homogenizing the number of iNKT cells added in the bioreactor at day 0 to 10 000/cm2 instead of 2×10⁶ total PBMCs (see FIG. 4A).

The expansion protocol was not improved by prolongating the expansion up to 21 days, with or without restimulation with PBMCs, alphagalcer and IL-15 at day 14 (see FIG. 4B).

The CD4⁻ iNKT cells obtained according to the above protocol were shown to be functional:
viability marker: >80% viable cells at the end of culture;
production of IFN-γ and IL-4 cytokines according to their CD4 phenotype;
expression of cytotoxicity molecules (CD95, NKG2D, NKp30, NKp44, NKp46);
ability to negatively regulate the activation of T lymphocytes stimulated in vitro by allogeneic dendritic cells;
ability to lyse human leukemic cell lines in vitro.
   4. Ability of the iNKT Cells Obtained According to the Present Invention to Control GVHD in a Humanized Xeno-GVHD Preclinical Model

NSG™ mice were irradiated (2 Gy total body irradiation) on day −1 and transplanted with 5×10⁶ human PBMC on day 0, without (PBMC alone group, n=4) or with 10⁶ human iNKT cells expanded as defined in the present invention (PBMC+iNKT group, n=9). The results are shown in FIG. 4. At day 60, all mice in the PBMC group have died of GVHD, while 5/9 mice in the PBMC+iNKT group were alive without GVHD.

These results suggest that the iNKT cells obtained according to the method of the present invention can be effectively used for preventing GVHD.

1: A method for obtaining Invariant Natural Killer T (iNKT) cells, said method comprising a step of culturing peripheral blood mononuclear cells (PBMC) in a culture medium comprising alpha-Galactosylceramide (alpha-GalCer) and Interleukin 15 (IL-15), wherein the expansion factor of iNKT cells is equal to or higher than 100.

2: The method according to claim 1, wherein the expansion factor of iNKT cells is equal to or higher than 500.

3: The method according to claim 1, wherein said method further comprises a step of screening and isolating CD4⁻ iNKT cells.

4: The method according to claim 1, wherein said culturing step is performed over 7 to 21 days, preferably over 14 days.

5: The method according to claim 1, wherein the concentration of IL-15 in the culture medium is comprised between 5 and 15 ng/mL, preferably around 10 ng/mL.

6: The method according to claim 1, wherein the concentration of alpha-GalCer in the culture medium is comprised between 50 and 150 ng/mL, preferably around 100 ng/mL.

7: The method according to claim 1, wherein the PBMCs are introduced in the culture medium at a concentration ranging from 1×10⁴ to 1×10⁷ cell/mL, preferably from 1×10⁵ to 2×10⁶ cells/mL.

8: The method according to claim 1, wherein said culture medium further comprises fetal bovine serum (FBS), human AB serum and/or glutamine.

9: The method according to claim 8, wherein the concentration of FBS and/or human AB serum is comprised between 1 and 8%, and the concentration of glutamine is comprised between 1 and 10 mM.

10: The method according to claim 1, wherein said culturing step is conducted at a temperature of 37° C. in presence of 5% of carbon dioxide (CO₂).

11: The method according to claim 1, wherein said PBMCs are firstly cultured in presence of alpha-GalCer prior adding IL-15.

12: The method according to claim 11, wherein IL-15 is introduced in the culture medium after 1 to 5 days of culture, preferably after 1 day of culture.

13: The method according to claim 1, wherein said method further comprises a step of replacing 20 to 80% of the culture medium used in the preceding steps with a culture medium comprising IL-15.

14: The method according to claim 13, wherein said step of replacing the culture medium is performed after 6 to 8 days of culture.

15. (canceled)
